(19) United States
(12) Patent Application Publication
(10) Pub. No.: US 2011/0098491 A1
(43) Pub. Date: Apr. 28, 2011

(54) Direct epoxidation process using alkanoic acid modifier

(76) Inventors: Bernard Cooker, Malvern, PA (US); Bi Le-Khac, West Chester, PA (US)

(21) Appl. No.: 12/589,775

(22) Filed: Oct. 28, 2009

(51) Int. Cl. C07D 301/06; C07D 301/04

(52) U.S. Cl. 549/533; 549/523

(57) ABSTRACT

The invention is a process for epoxidizing an olefin with hydrogen and oxygen in the presence of an alkanoic acid and a catalyst comprising a noble metal and a titanium zeolite, wherein the catalyst has not been reduced prior to epoxidation. This process surprisingly gives significantly improved productivity and reduced formation of unwanted propane compared to processes that do not use the alkanoic acid modifier.

### FIELD OF THE INVENTION

This invention relates to process for producing an epoxide by the reaction of an olefin, oxygen, and hydrogen.

### BACKGROUND OF THE INVENTION

Many different methods for the preparation of epoxides have been developed. Generally, epoxides are formed by the reaction of an olefin with an oxidizing agent in the presence of a catalyst. Ethylene oxide is commercially produced by the reaction of ethylene with oxygen over a silver catalyst. Propylene oxide is commercially produced by reacting propylene with an organic hydroperoxide oxidizing agent, such as ethylbenzene hydroperoxide or tert-butyl hydroperoxide. This process is performed in the presence of a solubilized molybdenum catalyst, see U.S. Pat. No. 3,351,635, or a heterogeneous titania on silica catalyst, see U.S. Pat. No. 4,367,342.

Besides oxygen and alkyl hydroperoxides, hydrogen peroxide is also a useful oxidizing agent for epoxide formation. U.S. Pat. Nos. 4,833,260 and 4,937,216, for example, disclose olefin epoxidation with hydrogen peroxide in the presence of a titanium silicate catalyst. U.S. Pat. Nos. 7,153,986 and 7,531,674 describe the epoxidation of propylene with hydrogen peroxide in the presence of an organic solvent and a crystalline titanosilicate having an MWW structure. Lunsford, et al. in J. Cat. 230 (2005), 313, teach the direct formation of H₂O₂ from H₂ and O₂ over a Pd/SiO₂ in ethanol or water that is acidified with H₂SO₄ or HCl.

Much current research is conducted in the direct epoxidation of olefins with oxygen and hydrogen. In this process, it is believed that oxygen and hydrogen react in situ to form an oxidizing agent. Many different catalysts have been proposed for use in the direct epoxidation process. Typically, the catalyst comprises a noble metal and a titanosilicate. For example, JP 4-352771 discloses the formation of propylene oxide from propylene, oxygen, and hydrogen using a catalyst containing a Group VIII metal such as palladium on a crystalline titanosilicate. U.S. Pat. No. 6,498,259 describes a catalyst mixture of a titanium zeolite and a supported palladium complex, where palladium is supported on carbon, silica, silica-alumina, titania, zirconia, and niobia. Other direct epoxidation catalyst examples include gold supported on titanosilicates, see for example PCT Intl. Appl. WO 98/00413. These catalysts are typically calcined in the presence of oxygen and then reduced prior to use.

One disadvantage of the described direct epoxidation catalysts is that they are prone to produce non-selective byproducts such as glycols or glycol ethers formed by the ring-opening of the epoxide product, alkane byproduct formed by the hydrogenation of olefin, or water formed by the decomposition of hydrogen peroxide or through direct formation from the elements. U.S. Pat. No. 6,008,388 teaches that the selectivity for the direct olefin epoxidation process is enhanced by the addition of a nitrogen compound such as ammonium hydroxide to the reaction mixture. U.S. Pat. No. 6,399,794 teaches the use of ammonium bicarbonate modifiers to decrease the production of ring-opened byproducts.

U.S. Pat. No. 6,005,123 teaches the use of phosphorus, sulfur, selenium or arsenic modifiers such as triphenylphosphine or benzothiophene to decrease the formation of unwanted propane. Reduced alkane byproduct formation is also taught in U.S. Pat. No. 7,595,410, which discloses the use of a lead-modified palladium-containing titanium zeolite, and U.S. Pat. No. 7,531,675 which discloses the use of a catalyst comprising a noble metal, lead, bismuth, and a titanium or vanadium zeolite.

As with any chemical process, it is desirable to attain still further improvements in the epoxidation methods. We have discovered a new process for the epoxidation of olefins.

### SUMMARY OF THE INVENTION

The invention is an olefin epoxidation process that comprises reacting olefin, oxygen, and hydrogen in the presence of alkanoic acid and a catalyst comprising a noble metal and a titanium zeolite, wherein the catalyst is not reduced prior to epoxidation. This process surprisingly gives significantly improved productivity and reduced formation of unwanted propane compared to processes that do not use the alkanoic acid modifier.

### DETAILED DESCRIPTION OF THE INVENTION

The process of the invention employs a catalyst that comprises a noble metal and a titanium zeolite. Titanium zeolites comprise the class of zeolitic substances wherein titanium atoms are substituted for a portion of the silicon atoms in the lattice framework of a molecular sieve. Such substances are well known in the art.

Particularly preferred titanium zeolites include the class of molecular sieves commonly referred to as titanium silicalites, particularly “TS-1” (having an MFI topology analogous to that of the ZSM-5 aluminosilicate zeolites), “TS-2” (having an MEL topology analogous to that of the ZSM-11 aluminosilicate zeolites), “TS-3” (as described in Belgian Pat. No. 1,001,038), and Ti-MWW (having a topology analogous to that of the MWW aluminosilicate zeolites). Titanium-containing molecular sieves having framework structures isomorphous to zeolite beta, mordenite, ZSM-48, ZSM-12, SBA-15, TUD, HMS, and MCM-41 are also suitable for use. TS-1 and Ti-MWW are particularly preferred. The titanium zeolites preferably contain no elements other than titanium, silicon, and oxygen in the lattice framework, although minor amounts of boron, iron, aluminum, sodium, potassium, copper and the like may be present.

The catalyst employed in the process of the invention also comprises a noble metal. The noble metal is preferably incorporated into the catalyst by supporting the noble metal on the titanium zeolite to form a noble metal-containing titanium zeolite, or alternatively, the noble metal may be first supported on a carrier such as an inorganic oxide, clay, carbon, or organic polymer resins, or the like, and then physically mixed with the titanium zeolite. There are no particular restrictions regarding the choice of noble metal compound used as the source of the noble metal. For example, suitable compounds include the nitrates, sulfates, halides (e.g., chlorides, bromides), carboxylates (e.g. acetate), oxides, and amine complexes of noble metals.

A preferred catalyst useful in the process of the invention is a noble metal-containing titanium zeolite. Such catalysts typically comprise a noble metal (such as palladium, gold, platinum, silver, iridium, ruthenium, osmium, or combinations thereof) supported on a titanium zeolite. Noble metal-containing titanium zeolites are well known in the art and are described, for example, in JP 4-352771 and U.S. Pat. Nos. 5,859,265 and 6,555,493, the teachings of which are incorporated herein by reference in their entirety. The noble metal-containing titanium zeolites may contain a mixture of noble metals. Preferred noble metal-containing titanium zeolites comprise palladium and a titanium zeolite; palladium, gold, and a titanium zeolite; or palladium, platinum, and a titanium zeolite.

The typical amount of noble metal present in the noble metal-containing titanium zeolite will preferably be in the range of from about 0.001 to 10 weight percent, more preferably 0.01 to 5 weight percent.

Another preferred catalyst useful in the process of the invention is a mixture comprising a titanium zeolite and a supported noble metal. The supported noble metal comprises a noble metal and a carrier. The carrier is preferably a porous material. Carriers are well-known in the art. For instance, the carrier can be inorganic oxides, clays, carbon, and organic polymer resins. Preferred inorganic oxides include oxides of Group 2, 3, 4, 5, 6, 13, or 14 elements. Particularly preferred inorganic oxide carriers include silica, alumina, silica-aluminas, titania, zirconia, niobium oxides, tantalum oxides, molybdenum oxides, tungsten oxides, amorphous titania-silica, amorphous zirconia-silica, amorphous niobia-silica, and the like. The carrier may be a zeolite, but is not a titanium zeolite. Preferred organic polymer resins include polystyrene, styrene-divinylbenzene copolymers, crosslinked polyethyleneimines, and polybenzimidizole. Suitable carriers also include organic polymer resins grafted onto inorganic oxide carriers, such as polyethylenimine-silica. Preferred carriers also include carbon. Particularly preferred carriers include carbon, titania, zirconia, niobia, silica, alumina, silica-alumina, tantalum oxides, molybdenum oxides, tungsten oxides, titania-silica, zirconia-silica, niobia-silica, and mixtures thereof.

Preferably, the carrier has a surface area in the range of about 1 to about 700 m²/g, most preferably from about 10 to about 500 m²/g. Preferably, the pore volume of the carrier is in the range of about 0.1 to about 4.0 mL/g, more preferably from about 0.5 to about 3.5 mL/g, and most preferably from about 0.8 to about 3.0 mL/g. Preferably, the average particle size of the carrier is in the range of about 0.1 μm to about 0.5 inch, more preferably from about 1 μm to about 0.25 inch, and most preferably from about 10 μm to about 1/16 inch. The preferred particle size is dependent upon the type of reactor that is used, for example, larger particle sizes are preferred for a fixed bed reaction. The average pore diameter is typically in the range of about 10 to about 1000 Å, preferably about 20 to about 500 Å, and most preferably about 50 to about 350 Å.

The supported noble metal also contains a noble metal. While any of the noble metals can be utilized (i.e., gold, silver, platinum, palladium, iridium, ruthenium, osmium), either alone or in combination, palladium, platinum, gold, a palladium/platinum, or a palladium/gold combination are particularly desirable. Palladium is most preferred.

Typically, the amount of noble metal present in the supported noble metal will preferably be in the range of from 0.001 to 10 weight percent, preferably 0.01 to 5 weight percent.

The catalyst useful in the process of the invention preferably contains lead and/or bismuth. Most preferably, the catalyst contains palladium, lead and bismuth. As with the noble metal, lead and/or bismuth may be supported on the titanium zeolite or, alternatively, the lead and/or bismuth may be first supported on a carrier then physically mixed with the titanium zeolite.

Preferably, the catalyst will contain 0.001 to 10 weight percent of the noble metal, 0.001 to 5 weight percent of lead, and/or 0.001 to 5 weight percent bismuth. Most preferably, the catalyst contains 0.01 to 5 weight percent of the noble metal, 0.01 to 2 weight percent of lead and/or 0.01 to 2 weight percent bismuth. Preferably, the weight ratio of noble metal to lead (bismuth) in the catalyst is in the range of 0.1 to 10. While the choice of lead or bismuth compound used as the lead or bismuth source in the supported noble metal is not critical, suitable compounds include carboxylates (e.g., acetate, citrate), halides (e.g., chlorides, bromides, iodides), oxyhalides (e.g., oxychloride), carbonates, nitrates, phosphates, oxides, and sulfides.

Any suitable method may be used for the incorporation of the noble metal and optional lead and/or bismuth into the catalyst. For example, the noble metal and optional lead and/or bismuth may be supported on the titanium zeolite or the carrier by impregnation, ion-exchange, or incipient wetness techniques with, for example, palladium tetraammine chloride. If lead and/or bismuth is used, the order of addition of noble metal and optional lead and/or bismuth to the titanium zeolite or the carrier is not considered critical. However, it is preferred to add the lead and/or bismuth compound at the same time that the noble metal is introduced.

After noble metal and optional lead and/or bismuth incorporation, the noble metal-containing titanium zeolite or supported noble metal is recovered. Suitable catalyst recovery methods include filtration and washing, rotary evaporation and the like. The catalyst is typically dried prior to use in epoxidation. The drying temperature is preferably from about 50° C. to about 200° C.

After noble metal-containing titanium zeolite or supported noble metal formation, the catalyst is preferably thermally treated in the presence of an oxygen-containing gas. The thermal treatment temperature is typically from about 20° C. to about 800° C. More preferably, the catalyst is thermally treated in the presence of an oxygen-containing gas at a temperature from about 200° C. to 700° C. The catalyst is not reduced prior to use in the epoxidation reaction.

In the epoxidation process of the invention, the catalyst may be used as a powder or as a large particle size solid. If a noble metal-containing titanium zeolite is used, the noble metal-containing zeolite may is be used as a powder but is preferably spray dried, pelletized or extruded prior to use in epoxidation. If spray dried, pelletized or extruded, the noble metal-containing titanium zeolite may additionally comprise a binder or the like and may be molded, spray dried, shaped or extruded into any desired form prior to use in epoxidation. The noble metal-containing titanium zeolite may also be encapsulated in polymer as described in U.S. Pat. No. 7,030,255, the teachings of which are incorporated herein by reference in their entirety. If a mixture of titanium zeolite and supported noble metal is used, the titanium zeolite and supported noble metal may be pelletized or extruded together prior to use in epoxidation. If pelletized or extruded together, the catalyst mixture may additionally comprise a binder or the like and may be molded, spray dried, shaped or extruded into any desired form prior to use in epoxidation. The catalyst mixture may also be encapsulated in polymer as described in U.S. Pat. No. 7,030,255.

The epoxidation process of the invention also employs an alkanoic acid. The alkanoic acid modifier will preferably be added to the reaction mixture along with a solvent. The concentration of alkanoic acid in the reaction mixture is preferably in the range of from about 1 mM to about 100 mM and most preferably from about 5 mM to about 15 mM. The alkanoic acid is preferably a lower alkanoic acid containing from 2 to 6 carbon atoms, such as acetic, propionic, or butyric acid. Acetic acid is particularly preferred.

The alkanoic acid may also be introduced into the epoxidation reaction mixture by adding the alkanoic acid to the catalyst prior to epoxidation. Preferably, the noble metal-containing titanium zeolite or supported noble metal is soaked or stirred in a solution of the alkanoic acid (e.g., glacial acetic acid), recovered, and dried, without calcination or reduction. The catalyst, containing alkanoic acid, may then be used in the epoxidation reaction.

The epoxidation process of the invention comprises contacting an olefin, oxygen, and hydrogen in the presence of the alkanoic acid and the catalyst. Suitable olefins include any olefin having at least one carbon-carbon double bond, and generally from 2 to 60 carbon atoms. Preferably the olefin is an acyclic alkene of from 2 to 30 carbon atoms; the process of the invention is particularly suitable for epoxidizing C₂-C₆ olefins. More than one double bond may be present, as in a diene or triene for example. The olefin may be a hydrocarbon (i.e., contain only carbon and hydrogen atoms) or may contain functional groups such as halide, carboxyl, hydroxyl, ether, carbonyl, cyano, or nitro groups, or the like. The process of the invention is especially useful for converting propylene to propylene oxide.

Oxygen and hydrogen are also required for the epoxidation process. Although any sources of oxygen and hydrogen are suitable, molecular oxygen and molecular hydrogen are preferred.

Epoxidation according to the invention is carried out at a temperature effective to achieve the desired olefin epoxidation, preferably at temperatures in the range of 0-250° C., more preferably, 20-100° C., and most preferably, 40-65° C. The molar ratio of hydrogen to oxygen can usually be varied in the range of H₂:O₂=1:10 to 5:1 and is especially favorable at 1:5 to 2:1. The molar ratio of oxygen to olefin is usually 5:1 to 1:20, and preferably 5:1 to 1:1.

A carrier gas may also be used in the epoxidation process. As the carrier gas, any desired inert gas can be used. Noble gases (such as helium, neon, and argon), nitrogen and carbon dioxide are suitable carrier gases. Saturated hydrocarbons with 1-8, especially 1-6, and preferably with 1-4 carbon atoms, e.g., methane, ethane, propane, and n-butane, are also suitable. Nitrogen and saturated C₁-C₄ hydrocarbons are preferred inert carrier gases. Mixtures of the listed inert carrier gases can also be used. The molar ratio of olefin to carrier gas is then usually in the range of 100:1 to 1:10 and especially 20:1 to 1:10.

Specifically in the epoxidation of propylene, propane can be supplied in such a way that, in the presence of an appropriate excess of carrier gas, the explosive limits of mixtures of propylene, propane, hydrogen, and oxygen are safely avoided and thus no explosive mixture can form in the reactor or in the feed and discharge lines.

Preferably, epoxidation is carried out in the liquid (or supercritical or subcritical) phase. It is advantageous to work at a pressure of 1-100 bars and in the presence of one or more solvents. Suitable solvents include any chemical that is a liquid under reaction conditions, including, but not limited to, oxygenated hydrocarbons such as alcohols, ethers, esters, and ketones, aromatic and aliphatic hydrocarbons such as toluene and hexane, nitriles such as acetonitrile, liquid CO₂ (in the supercritical or subcritical state), and water. Preferable solvents include liquid CO₂, nitriles, alcohols, ketones, water, and mixtures thereof. Preferred nitriles include acetonitrile and other nitriles with appreciable water solubility. Preferred alcohols include lower aliphatic C₁-C₄ alcohols such as methanol, ethanol, isopropanol, and tert-butanol, or mixtures thereof. Fluorinated alcohols can be used. Most preferably, the solvent is methanol, ethanol, isopropanol, and tert-butanol, water, or mixtures thereof. It is particularly preferable to use mixtures of the cited alcohols with water.

The process may be performed using a continuous flow, semi-batch or batch mode of operation. When a liquid reaction medium is used, the catalyst is preferably in the form of a suspension or fixed-bed.

If epoxidation is carried out in the liquid (or supercritical or subcritical) phase, it is advantageous to use a buffer. The buffer will typically be added to the solvent to form a buffer solution. The buffer solution is employed in the reaction to inhibit the formation of glycols or glycol ethers during epoxidation. Buffers are well known in the art. If a buffer is used, the alkanoic acid may be present in the epoxidation reaction as an equilibrium mixture of alkanoic acid and the corresponding alkanoate.

Buffers useful in this invention include any suitable salts of is oxyacids, the nature and proportions of which in the mixture, are such that the pH of their solutions may range from 3 to 10, preferably from 4 to 9 and more preferably from 5 to 8. Suitable salts of oxyacids contain an anion and cation. The anion portion of the salt may include anions such as phosphate, monohydrogenphosphate, dihydrogenphosphate, sulfate, carbonate, bicarbonate, citrate, borate, hydroxide, silicate, aluminosilicate, or the like. The cation portion of the salt may include cations such as ammonium, alkylammoniums (e.g., tetraalkylammoniums, pyridiniums, and the like), alkali metals, alkaline earth metals, or the like. Examples include NH₄, NBu₄, NMe₄, Li, Na, K, Cs, Mg, and Ca cations. More preferred buffers include alkali metal phosphate and ammonium phosphate buffers, including ammonium dihydrogen phosphate. Buffers may preferably contain a combination of more than one suitable salt. The buffer useful in this invention may also include the addition of ammonia gas to the reaction system or aqueous liquid ammonia solution to the reaction system. A combination of ammonium dihydrogen phosphate and aqueous ammonia is particularly preferred. Typically, the concentration of buffer in the solvent is from about 0.0001 M to about 1 M, preferably from about 0.001 M to about 0.3 M.

The following examples merely illustrate the invention. Those skilled in the art will recognize many variations that are within the spirit of the invention and scope of the claims.

### Example 1

### Catalyst Preparation

Ti-MWW Preparation:

Ti-MWW can be made according to Wu et al., J. Phys. Chem. B, 2001, 105, p. 2897.

A precursor gel is formed by combining and mixing fumed silica (180 g), boric acid (248.4 g), tetrabutyl orthotitanate (21.6 g), piperidine (547.5 g), and deionized (1026 g). A portion of the gel is crystallized in a 600-mL Parr reactor by heating at a temperature of 130° C. for 1 day, followed by 150° C. for 1 day, and finally at 170° C. for 10 days. Upon opening the reactor, a catalyst precursor is obtained as a white crystalline solid in a milky suspension. The catalyst precursor is collected by pressure filtration, rinsed at least twice with deionized water to give a final filtrate pH of approximately 9, and vacuum dried overnight.

To remove boron and extra-framework titanium, the catalyst precursor is treated by stirring the precursor in 4M HNO₃ at 80° C. for 16 hours (at a ratio of 1 g of catalyst precursor per 20 mL of acid solution). The solids are isolated by filtration, rinsed with deionized water until the filtrate pH was about 4, and vacuum dried. The solids are calcined in air in a muffle furnace by heating at 110° C. for 4 hours (after ramping at 10° C./min) and then at 530° C. for 6 hours (after ramping at 2° C./min), followed by cooling to room temperature to obtain Ti-MWW.

Pd—Bi—Pb/TiO₂ Preparation

Lead nitrate (2.08 g) and an aqueous solution of palladium nitrate (6.41 g, 20.64 wt. % Pd) are added to a solution of bismuth nitrate (0.9 g Bi(NO₃)₃ dissolved in 40 mL, 2.56 M solution of nitric acid, 16.6% by volume of 70% HNO₃) with mixing. The Pd—Bi—Pb solution is then added by incipient wetness to spray dried titania (60 g, 30 micron size, 40 m²/g, calcined in air at 700° C.). The solids are calcined in air in a muffle furnace by heating at 10° C./min to 110° C. for 4 hours and then at 2° C./min to 300° C. for 4 hours. These calcined solids are then washed with an aqueous sodium bicarbonate solution (20 mL, containing 0.9 g NaHCO₃, four times), followed by deionized water (20 mL, three times). The washed solids are calcined in a muffle furnace by heating at 10° C./min to 110° C. for 4 hours and then heating at 2° C./min to 600° C. for 4 hours to produce Pd—Bi—Pb/TiO₂ catalyst. The Pd—Bi—Pb/TiO₂ catalyst contains 1.6 wt. % Pd, 0.49 wt. % Bi, 1.6 wt. % Pb, and less than 100 ppm Na.

### Example 2

### Propylene Epoxidation Using Acetic Acid Modifier

To evaluate the performance of the catalysts prepared in Example 1 in the presence of acid modifiers, the epoxidation of propylene using oxygen and hydrogen is carried out. The following procedure is employed.

A 1000-cc stainless steel reactor is charged with a mixture of Pd—Bi—Pb/TiO₂ catalyst (0.6081 g). To condition the reactor prior to epoxidation, methanol solvent is fed continuously at 120 cc/hr, the reactor is charged to 520 psig with pure nitrogen fed continuously at 2300 cc/min, and the reactor temperature is maintained at 60° C. for 22.25 hours.

After conditioning the reactor, the reactor is charged with Ti-MWW powder (8.3449 g) and the methanol solvent is changed to an ammonium dihydrogen phosphate (ADHP) solution consisting of ADHP (10 mM concentration) in a tert-butyl alcohol (TBA) and water mixture (70% TBA, 30% water by weight) and the reactor is charged to 520 psig with a feed gas consisting of 1.79% hydrogen, 4.23% oxygen, 13.53% propylene and the balance nitrogen (volume %). The liquid feed is fed continuously at 150 cc/hr, and the pressure in the reactor is maintained at 520 psig via a backpressure regulator with the feed gases passed continuously through the reactor at 4898.6 cc/min (measured at 21.1° C. and one atmosphere pressure). The pH of the reaction mixture is adjusted to a pH of 6 by addition of aqueous ammonia. The reactor is stirred at 500 rpm, the reaction mixture is heated to 60° C. and the gaseous effluent is analyzed by an online GC every 2 hours and the liquid analyzed by an online GC simultaneously.

The epoxidation reaction is run continuously for 833 hours. The reaction conditions are varied over the life of the run. Table 1 shows the start time for the beginning of each new run and the change of conditions. The conditions for Comp. Run 2A are described above. In Comp. Run 2B, the pH is adjusted to 7. In Comp. Run 2C, the pH is adjusted to 8. In Comp. Run 2D, the pH is adjusted to 6.5 and the solvent is changed to a methanol-water mixture (80% MeOH and 20% water, by weight). In Comp. Run 2E, the pH is adjusted to 6 and the solvent is changed back to a 70 wt. % TBA-30 wt. % water mixture for the remainder of the 833-hour epoxidation reaction. In Comp. Run 2F, formic acid (10 mM) is added to the TBA-water mixture. In Run 2G, acetic acid (10 mM) is added to the TBA-water mixture. In Run 2H, acetic acid (10 mM) is still added and the reactor pressure is adjusted to 750 psig.

Propylene oxide and equivalents (“POE”), which include propylene oxide (“PO”), propylene glycol (“PG”), and propylene glycol methyl ethers (PMs), are produced during the reaction, in addition to propane formed by the hydrogenation of propylene. The results of the GC analyses are used to calculate the average productivity and average propane selectivities shown in the Table 1. The productivity and propane selectivities are averaged over the course of each run.

The epoxidation results (see Table 1) show that the presence of an alkanoic acid results in a significant increase in productivity and a significant decrease in propane formation, compared to runs without alkanoic acid or runs using formic acid.

TABLE 1
EFFECT OF ACETIC ACID ON PROPYLENE EPOXIDATION

| | Start of | | | | Propane |
|---|---|---|---|---|---|
| Run # | Run (hr) | Feed pH | Acid | Productivity¹ | Sel. (%)² |
| 2A* | 22.25 | 6 | — | 0.001 | 30 |
| 2B* | 72 | 7 | — | 0.001 | 40 |
| 2C* | 167 | 8 | — | 0.001 | 40 |
| 2D*³ | 241 | 6.5 | — | 0.008 | 45 |
| 2E* | 332 | 6 | — | 0.001 | 70 |
| 2F* | 427 | 6 | Formic | 0.001 | 70 |
| 2G | 597 | 6 | Acetic | 0.06 | 15 |
| 2H⁴ | 666 | 6 | Acetic | 0.04 | 15 |

¹Productivity = grams POE produced/gram of catalyst per hour.
²Propylene Selectivity = moles propane/(moles POE + moles propane) × 100.
³Solvent was 80% methanol and 20% water.
⁴Reactor pressure was raised to 750 psig.
*Comparative Example

1. A process for producing an epoxide comprising reacting an olefin, oxygen, and hydrogen in the presence of an alkanoic acid and a catalyst comprising a titanium silicalite, palladium, and one or more metals selected from the group consisting of lead, bismuth, and mixtures thereof, wherein the catalyst has not been reduced prior to epoxidation.

2. The process of claim 1 wherein the catalyst is a palladium-containing titanium zeolite that contains the one or more, metals.

3. (canceled)

4. (canceled)

5. The process of claim 1 wherein the palladium and one or more metals are supported on a carrier selected from the group consisting of carbon, titania, zirconia, niobia, silica, alumina, silica-alumina, tantalum oxides, molybdenum oxides, tungsten oxides, titania-silica, zirconia-silica, niobia-silica, and mixtures thereof.

6. The process of claim 1 wherein the olefin is a C₂-C₆ olefin.

7. The process of claim 1 wherein the reaction is conducted in a solvent selected from the group consisting of oxygenated hydrocarbons, aromatic and aliphatic hydrocarbons, chlorinated aromatic and aliphatic hydrocarbons, supercritical CO₂, water, and mixtures thereof.

8. The process of claim 7 wherein the solvent is selected from the group consisting of methanol, ethanol, isopropanol, tert-butanol, water, and mixtures thereof.

9. The process of claim 1 wherein the alkanoic acid is a C₂-C₆ alkanoic acid.

10. The process of claim 1 wherein the alkanoic acid is acetic acid.

11. The process of claim 1 wherein the titanium silicalite is TS-1 or Ti-MWW.

12. A process for producing propylene oxide comprising reacting propylene, hydrogen and oxygen in a solvent in the presence of acetic acid and a catalyst comprising a titanium silicalite, palladium, and one or more metals selected from the group consisting of lead, bismuth, and mixtures thereof, wherein the catalyst has not been reduced prior to epoxidation.

13. The process of claim 12 wherein the palladium and one or more metals are supported on a carrier selected from the group consisting of carbon, titania, zirconia, niobium oxides, silica, alumina, silica-alumina, tantalum oxides, molybdenum oxides, tungsten oxides, titania-silica, zirconia-silica, niobia-silica, and mixtures thereof.

14. (canceled)

15. The process of claim 12 wherein the solvent is selected from the group consisting of methanol, ethanol, isopropanol, tert-butanol, water, and mixtures thereof.
